(19) United States
(12) Patent Application Publication
(10) Pub. No.: US 2023/0076246 A1
(43) Pub. Date: Mar. 9, 2023

(54) METHOD AND SYSTEM FOR PREDICTING ANALYTE LEVELS

(71) Applicants: Yeda Research and Development Co. Ltd., Rehovot (IL); Mor Research Applications Ltd., Ramat-Gan (IL)

(72) Inventors: Eran SEGAL, Ramat-HaSharon (IL); Smadar SHILO, Rehovot (IL); Yotam AMAR, Rehovot (IL)

(73) Assignees: Yeda Research and Development Co. Ltd., Rehovot (IL); Mor Research Applications Ltd., Ramat-Gan (IL)

(21) Appl. No.: 17/800,245

(22) PCT Filed: Feb. 17, 2020
(86) PCT No.: PCT/IL2020/050178

§ 371 Date: Aug. 17, 2022

(51) Int. Cl. A61B 5/00; G06N 3/08; A61B 5/145

(52) U.S. Cl. CPC A61B 5/7275; G06N 3/08; A61B 5/7267; A61B 5/14532; A61B 5/4839

(57) ABSTRACT

A method of predicting an analyte level comprises receiving a time-ordered series of levels of the analyte, monitored over a time-period; feeding a trained neural network procedure with the monitored levels; and displaying, based on an output received from the procedure, a predicted level of the analyte in a future time. The procedure can comprise a plurality of layers, wherein for at least one pair of layers, a number of inter-layer connections within the pair is higher for later monitored levels than for earlier monitored levels.

### FIELD AND BACKGROUND OF THE INVENTION

The present invention, in some embodiments thereof, relates to medicine and, more particularly, but not exclusively, to a method and system for predicting analyte levels in a biological liquid, such as, but not limited to, glucose levels in the blood.

Various procedures are commonly employed to determine the level of substances of clinical or research significance which may be present in biological liquids such as blood, whole blood, urine, plasma, serum, sweat, saliva and other body liquids or homogenized tissues. Such substances are commonly referred to as analytes.

For example, in the management of diabetes and the attainment of a successful therapy there is a need to continuously monitor the blood glucose level, which may affect cognitive functioning. With respect to the brain, blood glucose levels influence and affect memory, awareness and attention. The consequences of reduced or elevated blood glucose levels on cognitive function are therefore more severe for subjects with poor glucose control such as individuals afflicted with diabetes. Hyperglycemia refers to a condition in which the blood glucose is too high, and the hyperglycemic subject is in danger of falling into coma. Hypoglycemia refers to a condition in which the blood glucose is too low, and the hypoglycemic subject is in danger of developing tissue damage in the blood vessels, eyes, kidneys, nerves, etc.

### SUMMARY OF THE INVENTION

According to an aspect of some embodiments of the present invention there is provided a method of predicting an analyte level in a biological liquid. The method comprises: receiving a time-ordered series of levels of the analyte, monitored over a time-period; feeding a trained neural network procedure with the monitored levels; and displaying, based on an output received from the procedure, a predicted level of the analyte in a future time. In various exemplary embodiments of the invention the procedure comprises a plurality of layers, wherein for at least one pair of layers, a number of inter-layer connections within the pair is higher for later monitored levels than for earlier monitored levels.

According to some embodiments of the invention the analyte comprises glucose.

According to some embodiments of the invention the method comprising receiving time-ordered series of dose levels of a drug administered to the biological liquid during the time-period, and feeding the dose levels to the procedure.

According to some embodiments of the invention the analyte comprises glucose and the drug comprises insulin.

According to some embodiments of the invention the time-ordered series is characterized by a frequency of at least 6 analyte levels per hour.

According to some embodiments of the invention the time-ordered series is characterized by a frequency of less than four analyte levels per hour, and the method comprises interpolating the time-ordered series to provide a plurality of interpolated analyte levels, and updating the time-ordered series using the interpolated analyte levels.

According to an aspect of some embodiments of the present invention there is provided a computer software product, comprising a computer-readable medium in which program instructions are stored, which instructions, when read by a data processor, cause the data processor to receive a time-ordered monitored levels of the analyte over a time-period and to execute the method as delineated above and optionally and preferably as further detailed below.

According to an aspect of some embodiments of the present invention there is provided a system for predicting an analyte level in a biological liquid. The system comprises a monitoring device configured for monitoring levels of the analyte in the biological liquid over a time-period, a data processor, and a communication device configured for transmitting the monitored levels to the data processor in a time-ordered manner In various exemplary embodiments of the invention the data processor is configured for receiving the monitored levels, for accessing computer-readable medium storing a trained neural network procedure, for feeding the procedure with the monitored levels, and for displaying, based on output received from the procedure, a predicted level of the analyte in a future time. In various exemplary embodiments of the invention the procedure comprises a plurality of layers, wherein for at least one pair of layers a number of inter-layer connections within the pair is higher for later monitored levels than for earlier monitored levels.

According to some embodiments of the invention the analyte is glucose.

According to some embodiments of the invention the system comprises an automatic drug administering device configured for administered a drug to the biological liquid during the time-period, and for transmitting to the data processor data pertaining to dosage of the administered drug as a time-ordered series of dose levels, wherein the data processor is configured for feeding the dose levels to the procedure.

According to some embodiments of the invention the time-period is selected such that the dose levels include basal dose levels but not bolus dose levels.

According to some embodiments of the invention the future time is before administration of a bolus dose level of the drug.

According to some embodiments of the invention the analyte comprises glucose and the drug comprises insulin.

According to some embodiments of the invention the time-ordered series is characterized by a frequency of at least 6 analyte levels per hour.

According to some embodiments of the invention the time-ordered series is characterized by a frequency of less than four analyte levels per hour, and the data processor is configured for interpolating the time-ordered series to provide a plurality of interpolated analyte levels, and updating the time-ordered series using the interpolated analyte levels.

According to some embodiments of the invention the communication device communicates wirelessly with the data processor.

According to some embodiments of the invention the data processor is a component of a server computer, and is configured to transmit the predicted level of the analyte to a mobile device having a display for displaying the predicted level of the analyte on the display.

According to some embodiments of the invention the data processor is a component of a mobile device having a display, and wherein the data processor is configured to display the predicted level of the analyte on the display.

According to some embodiments of the invention the time-period is from about 1 hour to about 6 hours.

According to some embodiments of the invention the future time is at least 10 minutes after an end of the time-period.

According to some embodiments of the invention the inter-layer connections are defined over a triangular weight matrix.

According to some embodiments of the invention at least one layer of the procedure, other than the at least one pair of layers, is a fully connected layer.

According to some embodiments of the invention at least one layer of the pair is a hidden layer.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flowchart diagram of a method suitable for predicting an analyte level in a biological liquid, according to exemplary embodiments of the present invention.

FIGS. 2A and 2B are schematic illustrations of a pair of fully-connected layers (FIG. 2A), and a pair of gradually-connected layers (FIG. 2B), according to exemplary embodiments of the present invention.

FIG. 3 is a schematic illustration of an artificial neural network procedure suitable for exemplary embodiments of the present invention.

FIG. 4 is a schematic illustration of a system for predicting an analyte level in a biological liquid, according to exemplary embodiments of the present invention.

FIGS. 5A and 5B are schematic illustrations of a Fully Connected Layer with input and output of size n and the weights matrix W∈M_{n×n} (FIG. 5A), and a Gradually Connected Layer with input of size n×1, output_rows=1, step_size=1 and the upper triangular block weights matrix W∈M_{n×n} (FIG. 5B).

FIGS. 6A-D are schematic illustration of a Gradually connected neural network optimized by the clarke error grid analysis, taking as input CGM data of one patient from the cohort and predicting his/her future glucose levels. (A) Four hours of historical CGM data and insulin dosage are taken from the patient CGM and insulin pump data as input for the model. The dashed line represents a desired glucose range of 70 mg/dl to 180 mg/dl. (B) GCN models with 4 GCLs receiving the data as input. The models are optimized using the CEG loss function in which a weight is deterministically chosen for each zone. The layer is described as the layer name, GCL1-4 and the number of neurons in the layer. (C) The output of the models, glucose predictions for 60 minutes prediction horizon, are presented for both AR and CGN models. The red arrow indicates the point of prediction generated by the GCN model using the 4 hours historical window presented in panel A as input. (D) Predictions generated by AR and CGN models are presented on the Clarke Error Grid. The red arrow indicates the point of prediction generated by the GCN model using 4 hours of historical data presented in panel A as input. AR—Autoregressive model, CEG—Clarke Error Grid, CGM—Continuous glucose monitoring, GCL—Gradually connected layer, GCN—Gradually connected neural network.

FIGS. 7A-G show analysis of the performances of our model (GCN3) compare to a baseline model (AR) on different groups of T1DM patients. FIG. 7A shows the percentage of predictions in zones C-E of the CEG for every patient in our cohort, the patients are sorted by AR C-E (%). FIGS. 7B-E show the percentage of predictions in zones C-E of the CEG for: patients in different age groups (FIG. 7B), different HbA1c % values (FIG. 7C), different Coefficient of variation of glucose values (FIG. 7D), and different percent of time in the hypoglycemic range (<70 mg %) (FIG. 7E). Significance was calculated using t-test, comparing the two different methods on each subgroup of patients. ns=Non significant, p>0.05, * p<0.05, **p<0.01, ***p<0.001. FIGS. 7F-G show root means square error for patients with different Coefficient of variation of glucose values (FIG. 7F), and different percent of time in the hypoglycemic range (<70 mg %) (FIG. 7G). AR—Autoregressive model, CV—Coefficient of variation, CEG—Clarke Error Grid, T1DM—Type 1 diabetes mellitus, RMSE-Root means square error.

### DESCRIPTION OF SPECIFIC EMBODIMENTS OF THE INVENTION

The present invention, in some embodiments thereof, relates to medicine and, more particularly, but not exclusively, to a method and system for predicting analyte levels in a biological liquid, such as, but not limited to, glucose levels in the blood.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

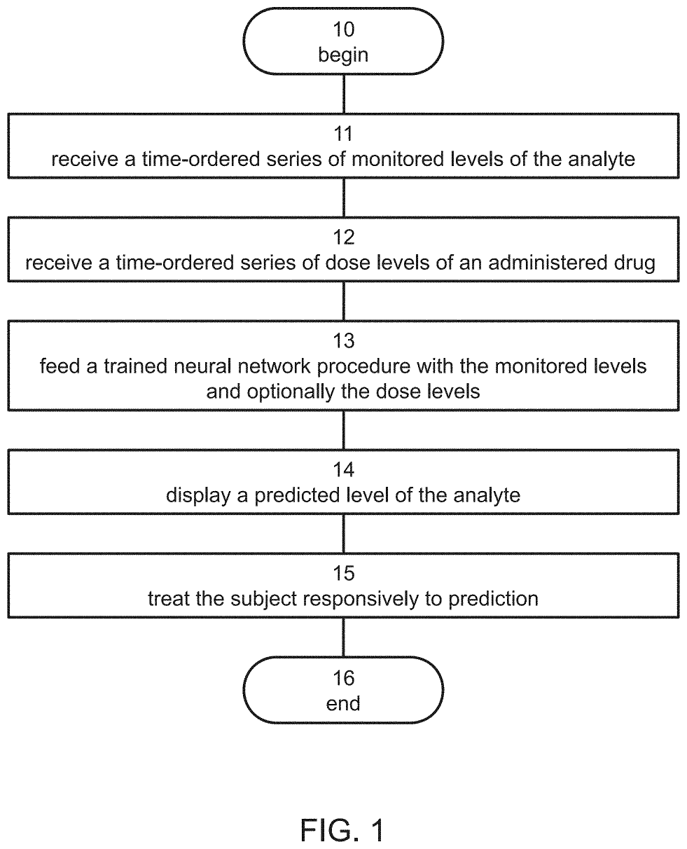

FIG. 1 is a flowchart diagram of a method suitable for predicting an analyte level in a biological liquid, according to various exemplary embodiments of the present invention. It is to be understood that, unless otherwise defined, the operations described hereinbelow can be executed either contemporaneously or sequentially in many combinations or orders of execution. Specifically, the ordering of the flowchart diagrams is not to be considered as limiting. For example, two or more operations, appearing in the following description or in the flowchart diagrams in a particular order, can be executed in a different order (e.g., a reverse order) or substantially contemporaneously. Additionally, several operations described below are optional and may not be executed.

At least part of the operations described herein can be can be implemented by a data processing system, e.g., a dedicated circuitry or a general purpose computer, configured for receiving data and executing the operations described below. At least part of the operations can be implemented by a cloud-computing facility at a remote location. The data processing system or cloud-computing facility can serve, at least for part of the operations as an image processing system, wherein the data received by the data processing system or cloud-computing facility include image data.

Computer programs implementing the method can commonly be distributed to users on a distribution medium such as, but not limited to, a flash memory, CD-ROM, or a remote medium communicating with a local computer over the internet. From the distribution medium, the computer programs can be copied to a hard disk or a similar intermediate storage medium. The computer programs can be run by loading the computer instructions either from their distribution medium or their intermediate storage medium into the execution memory of the computer, configuring the computer to act in accordance with the method. All these operations are well-known to those skilled in the art of computer systems.

The method can be embodied in many forms. For example, it can be embodied on a tangible medium such as a computer for performing the method steps. It can be embodied on a computer readable medium, comprising computer readable instructions for carrying out the method steps. In can also be embodied in electronic device having digital computer capabilities arranged to run the computer program on the tangible medium or execute the instruction on a computer readable medium.

The biological liquid is typically a body liquid of a subject under monitoring, and can be blood, whole blood, urine, plasma, serum, sweat, saliva or any other body liquid or homogenized tissue. The analyte whose level is to be predicted by the technique of the present embodiments can be glucose, cholesterol, ketone bodies, acetyl choline, amylase, bilirubin, chorionic gonadotropin, creatine kinase (e.g., CK-MB), creatine, DNA, fructosamine, glutamine, growth hormones, hormones, ketones, lactate, peroxide, prostate-specific antigen, prothrombin, RNA, thyroid stimulating hormone, troponin and the like. Preferably, the analyte is glucose.

The method begins at 10 and continues to 11 at which a time-ordered series of levels of the analyte is received. The levels of the analyte are preferably monitored over a time-period. The time-period can be from about 1 hour to about 6 hours, e.g., about 4 hours. Other values for the time-period, including less than one hour and more than six hours are also contemplated.

The levels of the analyte are typically read from a computer-readable medium. The levels can also be streamed over a communication network, and stored in a buffer of a computer-readable medium accessible by the method. The levels of the analyte are typically monitored using a monitoring device arranged to sense the analyte and transmit its levels continuously or repeatedly. The monitoring device is typically attached to a mammalian subject. The monitoring device can monitor the level of the analyte in an invasive or non-invasive manner. For example, when the analyte is glucose, the monitoring device can be a glucose monitoring device. As a representative example, the device can include a subcutaneous sensor that measure the level of glucose in the tissue and sends this information over a communication network to a monitor that stores the results. Such devices are commercially available, for example, from Medtronic, Inc. of Minneapolis, from Dexcom, San Diego, Calif., and from Abbott Laboratories, Illinois. Other representative examples for monitoring devices are found in U.S. Pat. Nos. 6,949,070, and 7,963,917, and U.S. Published Application No. 20160192867, the contents of which are hereby incorporated by reference.

In some embodiments of the present invention the monitoring device transmits the monitored levels of the analyte to a data processor that is configured to carry-on the operations described below, or to a computer-readable medium accessible by a data processor that is configured to carry-on the operations described below.

The time-ordered series of monitored analyte levels is optionally and preferably characterized by a frequency of at least 6 analyte levels per hour, more preferably at least 12 analyte levels per hour, more preferably at least 24 analyte levels per hour, more preferably at least 48 analyte levels per hour, e.g., about one analyte level per minute, or more. In some cases, the time-ordered series is characterized by a frequency of less than five or less than four or less than three analyte levels per hour, e.g., two or less analyte levels per hour. In this case the method optionally and preferably interpolates the time-ordered series to provide a plurality of interpolated analyte levels, and updates the time-ordered series using the interpolated analyte levels.

The method optionally and preferably proceeds to 12 at which the method receives a time-ordered series of dose levels of a drug that is administered to the biological liquid during the time-period. The drug is optionally and preferably administered automatically using an automatic drug administering device, which may include, for example, an infusion pump for subcutaneously drug delivery. Automatic drug administering devices are commercially available, for example, from Medtronic, Inc., Minneapolis, from Animas Technologies Ltd., Pennsylvania, and from Insulet Corporation, Massachusetts.

The type of drug typically corresponds to the type of monitored analyte. For example, the drug can include a pharmaceutically active agent that alters, directly or indirectly, the level of the monitored analyte in the biological liquid. Representative examples of types of drugs that can be administered including, without limitation, antidiabetic agents such as insulin, sulfonylureas, biguanides (such as metformin) α-glucosidase inhibitors (such as acarbose), and peroxisome proliferator-activater receptor γ agonists such as the glitazones (thiazolidinediones such as pioglitazone, troglitazone, MCC-555, and BRL49653); cholesterol lowering agents such as HMG-CoA reductase inhibitors (lovastatin, simvastatin and pravastatin, fluvastatin, atorvastatin, and other statins), sequestrants (cholestyramine, colestipol and a dialkylaminoalkyl derivatives of a cross-linked dextran), nicotinyl alcohol nicotinic acid or a salt thereof, proliferator-activater receptor α agonists such as fenofibric acid derivatives (gemfibrozil, clofibrat, fenofibrate and benzafibrate), and probucol.

In some embodiments of the present invention the automatic drug administering device transmits the dose levels of the drug to the data processor, or to a computer-readable medium accessible by the data processor.

The method proceeds to 13 at which an artificial neural network procedure is fed with the monitored levels of the analyte, and, when operation 12 is executed, the artificial neural network procedure is also fed with the dose levels.

In some embodiments of the present invention the method feeds the artificial neural network procedure with a portion of the series received at 11, wherein the portion is selected in accordance with the dose levels received 12. For example, it was found by the inventors that more accurate results are obtained, when the levels of the analyte are levels that are monitored before a bolus administration of the drug. Thus, according to various exemplary embodiments of the present invention the portion of the series is selected such that the time-period associated with the portion ends before the administration of a bolus of the drug. Thus, in these embodiments, the method feeds the artificial neural network procedure with data over a time-period in which the dose levels include basal dose levels but not bolus dose levels.

As a representative example, suppose that for a given time-period Δt, the method receives at 12 a time-ordered series D of m dose levels, respectively corresponding to m time-points at which the drug was administered, where the series D includes both entries corresponding to basal dose levels and entries corresponding to bolus dose levels. In this case, the method optionally and preferably truncates one or more entries from the beginning and/or end of D to provide a truncated series D′ of m′<m dose levels which include basal dose levels but not bolus dose levels. Suppose further that for the given time-period Δt, the method receives at 11 a time-ordered series A of n monitored analyte levels respectively corresponding to n time-points at which the levels are monitored. According to preferred embodiments of the invention, the method also truncates one or more entries from A to provide a truncated series A′ of n′<n monitored analyte levels, wherein the truncation of A is executed such that the time-period spanning from the first to the last dose level of D′ matches the time-period spanning from the first to the last monitored analyte level of A′. At 13, the method then feeds the entries of the truncated series to the artificial neural network.

Artificial neural networks are a class of computer implemented techniques that are based on a concept of inter-connected “artificial neurons,” also abbreviated “neurons.” In a typical artificial neural network, the artificial neurons contain data values, each of which affects the value of a connected artificial neuron according to connections with pre-defined strengths, and whether the sum of connections to each particular artificial neuron meets a pre-defined threshold. By determining proper connection strengths and threshold values (a process referred to as training), an artificial neural network can achieve efficient recognition of rules in the data. The artificial neurons are oftentimes grouped into interconnected layers, the number of which is referred to as the depth of the artificial neural network. Each layer of the network may have differing numbers of artificial neurons, and these may or may not be related to particular qualities of the input data. Some layers or sets of interconnected layers of an artificial neural network may operate independently from each other. Such layers or sets of interconnected layers are referred to as parallel layers or parallel sets of interconnected layers.

In some embodiments of the present invention the monitored levels of the analyte and the dose levels of the drug are fed to parallel sets of interconnected layers, so that at least part of the processing of the drug levels is independent of the monitored levels of the analyte. Alternatively, the monitored levels of the analyte and the dose levels of the drug can be combined to one vector that is fed to a set of interconnected layers.

The basic unit of an artificial neural network is the artificial neuron. It typically performs a product of its input and a weight. The input is given, while the weights are learned during the training phase and are held fixed during the validation or the testing phase. Bias may be introduced to the computation by concatenating a fixed value of 1 to the input vector creating a slightly longer input vector x, and increasing the dimensionality of w by one. The scalar product is typically followed by a non-linear activation function σ: [symbol] → [symbol] . Many types of activation functions that are known in the art, can be used in the artificial neural network of the present embodiments, including, without limitation, Binary step, Soft step, TanH, ArcTan, Softsign, Inverse square root unit (ISRU), Rectified linear unit (ReLU), Leaky rectified linear unit, Parametric rectified linear unit (PReLU), Randomized leaky rectified linear unit (RReLU), Exponential linear unit (ELU), Scaled exponential linear unit (SELU), S-shaped rectified linear activation unit (SReLU), Inverse square root linear unit (ISRLU), Adaptive piecewise linear (APL), SoftPlus, Bent identity, SoftExponential, Sinusoid, Sinc, Gaussian, Softmax and Maxout. In some embodiments of the present invention ReLU or a variant thereof (e.g., PReLU, RReLU, SReLU) is used.

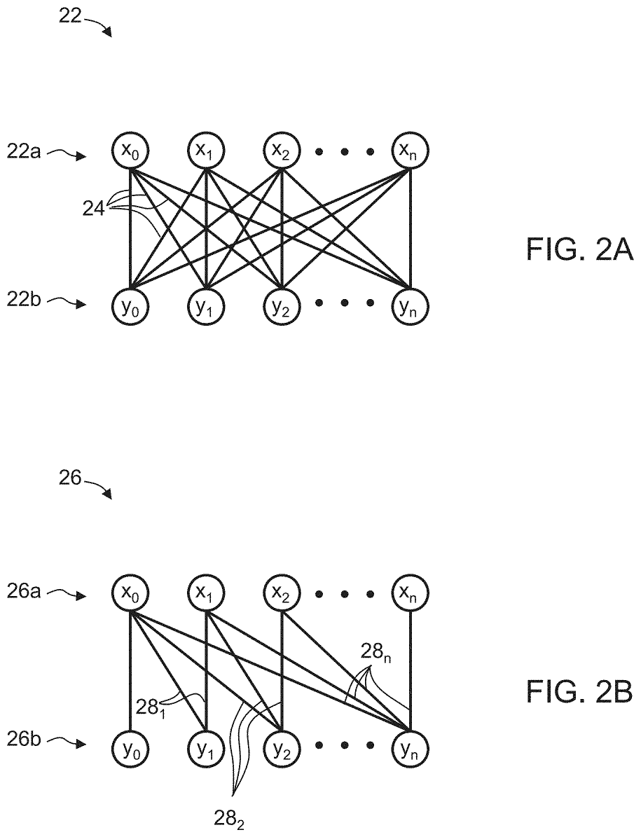

Known in the art are artificial neural networks which include fully-connected layers. In such layers, every neuron of the layer is connected by an inter-layer connection 24 to every neuron of the successive layer. In other words, the input of every neuron in the successive layer consists of a combination (e.g., a sum) of the activation values (the values after the activation function) of all the neurons in the previous layer. FIG. 2A is a schematic illustrations of a pair 22a, 22b of fully-connected layers, including a layer 22a and a layer 22b where layer 22a serves as the input to layer 22b. Each layer 22a, 22b includes a plurality of neurons. In practice the layers may include hundreds of neurons. As shown, each neuron y₀, . . . yₙ of layer 22b receives its input from each of the neurons x₀, . . . xₙ, of layer 22a. The activation values of the neuron y₀, yₙ are calculated according to the equation y=xW, where y is a vector whose components are y₀, . . . yₙ, x a vector whose components are x₀, . . . xₙ, and W is a weight matrix:

$$W = ( \begin{matrix}{w}_{0 , 0} & … & {w}_{0 , n} \\ ⋮ & ⋱ & ⋮ \\ {w}_{n , 0} & … & {w}_{n , n}\end{matrix} )$$

Unlike conventional fully-connected layers, the artificial neural network used according to preferred embodiments of the present invention includes at least one pair of layers in which the number of inter-layer connections within the pair is higher for later monitored analyte levels (and optionally dose levels, if operation 12 is employed), than for earlier monitored analyte levels (and optionally dose levels, if operation 12 is employed). This embodiment is illustrated in FIG. 2B, which is a schematic illustration of a pair 26a, 26b of gradually-connected layers, including a layer 26a and a layer 26b, where layer 26a serves as the input to layer 26b. Each of layers 26a, 26b includes a plurality of neurons. In practice the layers may include hundreds of neurons.

The neurons are arranged in a time-ordered manner within the layers, so that neurons with larger indices correspond to monitored analyte or dose levels at later times (e.g., x₀ corresponds to analyte level measured at time t₀, x₁ corresponds to analyte level measured at time t₁, and so on, where tₙ>tₙ₋₁> . . . > t₀). Unlike the pair 22 of fully connected layers illustrated in FIG. 2A, not all the neuron y₀, . . . yₙ of layer 26b receive their input from all the neurons x₀, . . . xₙ, of layer 26a. Rather, for a given two neurons yᵢ and yⱼ of layer 26b, where i>j, the number of inter-layer connections 28ᵢ for yᵢ is larger than the number of inter-layer connections 28ⱼ for yⱼ. For example, as illustrated in FIG. 2B, the number of inter-layer connections 28₁ for y₁ is 2, the number of inter-layer connections 28₂ for y₂ is 3, and the number of inter-layer connections 28n for yₙ is n+1. Formally, this can be achieved by imposing zeroes for a portion of the weights in the weight matrix W. For example, in some embodiments, the matrix W is a triangular matrix, with zero matrix elements below or above the main diagonal:

$$W = ( \begin{matrix}{w}_{0 , 0} & {w}_{0 , 1} & … & {w}_{0 , n} \\ 0 & {w}_{1 , 1} & … & {w}_{1 , n} \\ ⋮ & 0 & ⋱ & ⋮ \\ 0 & … & 0 & {w}_{n , n}\end{matrix} )$$

The inventors found that the use of gradually-connected layers according to some embodiments of the present invention is advantageous since it provides predictions with higher accuracy. The rational for selecting gradually-connected layers is that they provide higher expressive power to more recent data.

In some embodiments of the invention, the neural network comprises a plurality of hidden gradually-connected layers arranged consecutively such that in each pair of gradually-connected layers the number of inter-layer connections gradually increases similarly to the inter-layer connections described above with respect to pair 26a, 26b.

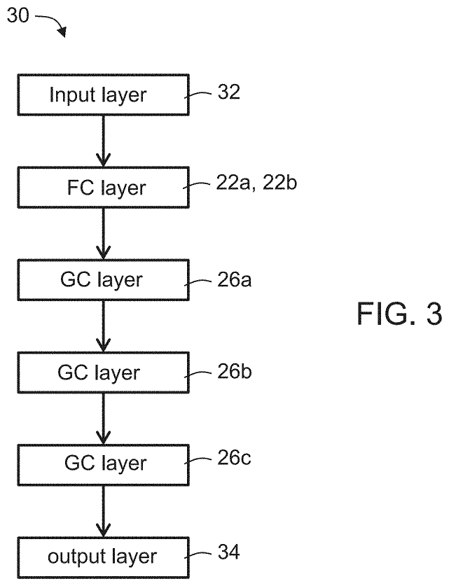

A representative example of an artificial neural network procedure 30 suitable for the present embodiments is illustrated in FIG. 3. Neural network procedure 30 comprises an input layer 32 for receiving the monitored analyte levels and optionally also the dose levels. Alternatively, neural network procedure 30 can comprise a separate input layer for receiving the monitored analyte levels, and a separate input layer for receiving the dose levels. Optionally and preferably, neural network procedure 30 comprises one or more fully-connected (FC) layers, such as layers 22a, 22b described above. Network procedure 30 comprises a plurality of gradually-connected (GC) layers 26a, 26b, 26c, as further detailed hereinabove, and an output layer 34. Although FIG. 3 shows three GC layers, this need not necessarily be the case since neural network procedure 30 can include any number of GC layers (e.g., less than 3 layers or more than 3 GC layers). For clarity of presentation, the inter-layer connections in FIG. 3 are illustrated by single arrow, but the ordinarily skilled person, provided with the details described herein, would appreciate that there is a plurality of inter-layer connections between any pair of layers in neural network procedure 30. The FC layers (when employed) and the GC layers are referred to herein as “hidden layers”, since they do not communicate directly from outside the procedure.

The computation of activation values for the neurons of the layers of neural network 30 continues through the various layers, for example, using weight matrixes, as further detailed hereinabove. Once the activation values of the final hidden layer (layer 26c, in the present Examples) are computed, they are combined to output layer 34. Typically, some concatenation of neuron values is executed before the output layer 34. At this point, the output of neural network procedure 30 can be extracted from the values in the output layer 34.

In the present embodiments, the output layer 34 of the neural network preferably provides a prediction of the level of the analyte in a future time, namely at a time point that is beyond the time point associated with the last monitored level of the analyte in the time-ordered series received at 11.

The neural network procedure used according to some embodiments of the present invention is a trained machine learning procedure. A neural network procedure can be trained according to some embodiments of the present invention by feeding a neural network training program with a time-ordered series of monitored levels of the analyte, and optionally and preferably also time-ordered series of dose levels of the drug. The neural network training program can use more recent levels as output test data enacting predictions that are to be provided at the output layer, and less recent levels as input test data enacting data to be fed to the input layer. The neural network training program then calculates the weight matrices that optimize the relation between the input test data and the output test data. Once the weights are calculated, the neural network training program generates a trained neural network procedure which can then be used without the need to re-train it.

The Examples section that follows describes a neural network training program that was used to generate a trained neural network procedure that provides, at its output layer, predictions of the level of the analyte in a future time.

In some embodiments of the present invention the neural network procedure is trained to provide a prediction of the level of the analyte in a future time which is at least 10 minutes or at least 15 minutes or at least 20 minutes or at least 25 minutes or at least 30 minutes or after the end of the time-period.

Referring again to FIG. 1, the method proceeds to 14 at which, based on the output received from the neural network procedure, a predicted level of the analyte is displayed.

The method optionally and preferably proceeds to 15 at which the subject is treated for reducing or increasing the level of the analyte in the body liquid, responsively to the predicted level. Typically, the method compares the predicted level to one or more predetermined thresholds, and treat the subject based on the comparison. The treatment can in various exemplary embodiments of the invention be executed by the automatic drug administering device, but treatment by other devices, or by hospitalization are also contemplated.

For example, when the analyte is blood glucose, the predicted glucose level is compared to a first threshold and/or second threshold, where the second threshold is higher than the first threshold. If the predicted glucose level is less than the first threshold, the method can treat the subject with glucose, for example, by administering dextrose, wherein the dosage and/or rate of administration of the glucose is selected based on the comparison so as to prevent the blood glucose level from decreasing to the predicted level. If the predicted glucose level is more than the second threshold, the method can treat the subject with an antidiabetic agent, such as, but not limited to, one or more of the aforementioned antidiabetic agents, wherein the dosage and/or rate of administration of the antidiabetic agent is selected based on the comparison so as to prevent the blood glucose level from reaching the predicted level.

The method ends at 16.

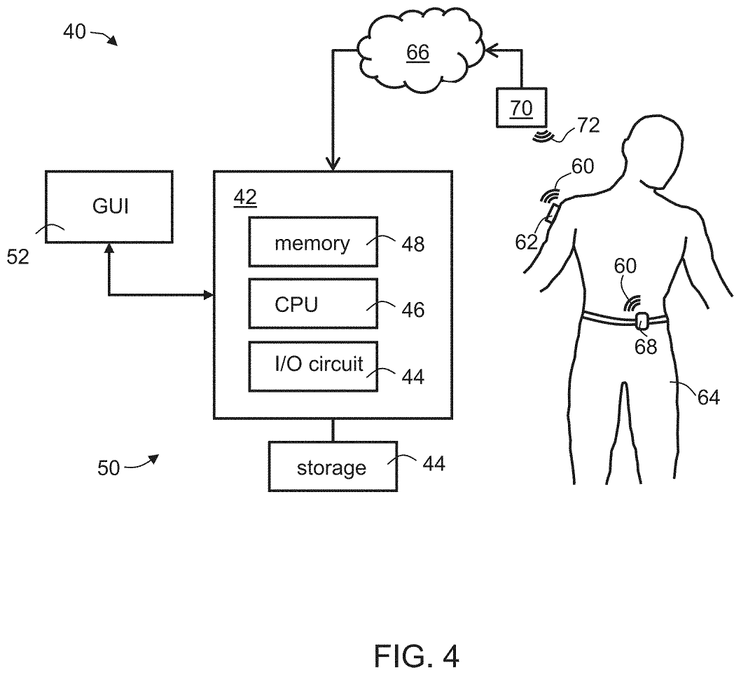

FIG. 4 is a schematic illustration of a system 40 for predicting an analyte level in a biological liquid, according to some embodiments of the present invention.

System 40 comprises a hardware data processor 42, which typically comprises an input/output (I/O) circuit 44, a hardware central processing unit (CPU) 46 (e.g., a hardware microprocessor), and a hardware memory 48 which typically includes both volatile memory and non-volatile memory. CPU 46 is in communication with I/O circuit 44 and memory 48. In some embodiments of the present invention data processor 42 is a component of a computer 50. Computer 50 can comprises a graphical user interface (GUI) 52 in communication with processor 42. I/O circuit 44 preferably communicates information in appropriately structured form to and from GUI 52. In some embodiments of the present invention computer 50 is a server computer, such as, but not limited to, a part of a cloud computing resource of a cloud computing facility. In some embodiments of the present invention data processor 42 is a component of a mobile device, in which case computer 50 is the mobile device, e.g., a smartphone device, a tablet device or a smartwatch device.

System 40 can further comprise a monitoring device 62 for monitoring levels of the analyte, as further detailed hereinabove. In some embodiments of the present invention monitoring device 62 is mounted on the skin of a subject 64 and is arranged for monitoring levels of the analyte in a body liquid (e.g., the blood) of subject 64. In some embodiments of the present invention system 40 also comprises an automatic drug administering device 68 for automatically administering a drug to subject 64. Typically, device 68 is also mounted on the skin of subject 68. For example, device 68 can be includes an automatic pump and a microneedle for subcutaneously drug delivery. In some embodiments of the present invention both devices 62 and 68 are encapsulated in the same encapsulation, and in some embodiments of the present invention devices 62 and 68 are provided separately from each other.

Monitoring device 62 and/or drug administering device 68 can serve as transmitters that communicate information to computer 50 via a wired or wireless communication. For example, computer 50 and devices 62 and/or 68 can communicate via a network 66, such as a Bluetooth network, a Wireless Fidelity (WiFi) network, a wireless local area network (WLAN), a ZigBee network, a local area network (LAN), a wide area network (WAN), or the Internet. Preferably, the communication is of the wireless type, in which case device 62 and/or 68 is configured to generate and radiate an electromagnetic signal 60 encoding the respective series. In these embodiments, system 40 can comprise a wireless network transceiver 70 configured to wirelessly receive signal 60 and transmit it to computer 50 over network 66. Alternatively, network transceiver 70 can be part of computer 50 in which signal 60 is transmitted directly to computer 70. Typically, wireless network transceiver 70 configured transmits a broadcast signal 72 which is received by devices 62 and 68 as known in the art of wireless communication.

GUI 52 and processor 42 can be integrated together within the same housing (e.g., when computer 50 is a mobile device) or they can be separate units communicating with each other. GUI 52 can optionally and preferably be part of a system including a dedicated CPU and I/O circuits (not shown) to allow GUI 52 to communicate with processor 42. Processor 42 issues to GUI 52 graphical and textual output generated by CPU 46. Processor 42 can also receive from GUI 52 signals pertaining to control commands generated by GUI 52 in response to user input. GUI 52 can be of any type known in the art, such as, but not limited to, a keyboard and a display, a touch screen, and the like. In preferred embodiments, GUI 52 is a GUI of a mobile device such as a smartphone, a tablet, a smartwatch and the like.

Computer 50 can further comprise one or more computer-readable storage media 54. Medium 54 is preferably a non-transitory storage medium storing computer code instructions as further detailed herein, and processor 42 executes these code instructions. The code instructions can be run by loading the respective code instructions into the execution memory 48 of processors 42. Storage medium 44 preferably also stores a trained artificial neural network, such as, but not limited to, network 30 as further detailed hereinabove.

In operation, processor 42 of computer 50 receives the time-ordered series of monitored levels of the analyte from device 62, and optionally and preferably also the time-ordered series of dose levels from device 68. Processor 42 optionally and preferably executes code instructions to store the received time-ordered series, at least temporarily, in memory 48 or storage medium 44. Processor 42 executes code instructions to access storage medium 44 storing the trained neural network procedure, and to feed the procedure with data including the monitored levels and optionally the dose levels. The trained neural network procedure processes the data through the various layers of the trained neural network using the pre-calculated weights obtained during the training as further detailed hereinabove. The trained neural network provides a prediction of the analyte at its output layer as further detailed hereinabove. Processor 42 executes code instructions to display on GUI 52 the predicted level of the analyte in a future time, based on the output received from the trained neural network procedure.

When GUI 52 is a GUI of a mobile device, the CPU circuit of the mobile device can serve as processor 42 and can execute the code instructions described herein. Alternatively, GUI 52 can be a GUI of a mobile device, and processor 42 can be a processor of a remote server computer, in which case processor 42 and the mobile device can communicate with each other over the network 66. In these embodiments, the code instructions described herein are executed by the processor of the remote server computer, and the predicted level is transmitted to the mobile device for displaying the predicted levels by the CPU or GPU of the mobile device on GUI 52.

In some embodiments of the present invention processor 42 executes code instructions for transmitting an operation signal to device 68 to increase or decrease the dosage of the administered drug, and/or to treat subject by another agent or drug. Typically, the code instructions instruct processor 42 to compare the predicted level to one or more predetermined thresholds, and treat the subject based on the comparison.

For example, when the analyte is blood glucose, processor 42 compares it to the first and/or second thresholds, as further detailed hereinabove. If the predicted glucose level is less than the first threshold, processor 42 can signal device 68 to reduce the dosage of antidiabetic agent that device 68 administer to subject 64, or to administer subject 64 with glucose as further detailed hereinabove. In various exemplary embodiments of the invention processor 42 calculates the dosage and/or rate of administration based on the comparison so as to prevent the blood glucose level from decreasing to the predicted level. If the predicted glucose level is more than the second threshold, processor 42 can signal device 68 to increase the dosage of the antidiabetic agent that device 68 administer to subject 64. In various exemplary embodiments of the invention processor 42 calculates the dosage and/or rate of administration of the antidiabetic agent based on the comparison so as to prevent the blood glucose level from reaching the predicted level.

As used herein the term “about” refers to ±10%.

The word “exemplary” is used herein to mean “serving as an example, instance or illustration.” Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments.” Any particular embodiment of the invention may include a plurality of “optional” features unless such features conflict.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Prediction of Glucose Levels in Patients with Type 1 Diabetes

Accurate prediction of blood glucose levels in patients with type 1 diabetes mellitus (T1DM) is advantageous both for their glycemic control and for the development of reliable closed loop systems. This Example demonstrates a novel computational model that outperforms traditional prediction methods.

T1DM is one of the most common chronic diseases in children and adolescents. In the past decade, there is an alarming increase in the incidence of T1DM worldwide, which is more prominent in younger children. Management of T1DM is a challenge for both patients and caregivers. Although intensive insulin therapy can prevent microvascular complications and cardiovascular morbidity, it is associated with higher risk of hypoglycemia, weight gain, and an increased burden of self-management. Despite substantial progress in diabetes technologies over the past decades, recent studies demonstrate that clinical management of T1DM is still lacking, particularly in children and adolescents, with many patients not meeting their glycemic goals.

The development of continuous glucose monitoring (CGM) devices, which measure glucose in the interstitial fluid continuously was an important milestone in diabetes monitoring technology and enabled the development of new treatment strategies such as closed loop systems, also termed “artificial pancreas” (AP). These systems consist of three components: a CGM, an infusion pump, and a dosing algorithm. Prediction of glucose levels is a challenging task due to a high intra- and inter-patient variability, and the influence of many factors such as food consumption, insulin dosage, physical activity, and emotional status. Most of the known control algorithms for AP systems, including Model Predictive Controllers (MPC) (11), Proportional Integral Derivative Controllers (PID) (12) and fuzzy-logic based controllers (13), use naïve, mostly linear, predictors of blood glucose levels, and rely mostly on medical logic, rather than on data-driven approaches. In addition, many of these computational models were constructed and evaluated using in silico data, generated by a T1DM Simulator (14,15), and were not evaluated using real life data.

The data utilized in this Example included real-life retrospective continuous glucose monitoring (CGM) data from 141 patients with T1DM, totaling 9,083 CGM connection days (1,592,506 glucose measurements), and in silico data generated by the UVA/Padova T1DM Simulator. The clinical accuracy, measured by the percentage of time in each of the Clarke Error Grid (CEG) zones, of predictions done by the technique of the present embodiments was compared to traditional methods.

### Methods

Data Acquisition

The database used for training and evaluation of the computational models included retrospective CGM and insulin pumps data of patients with T1DM who visited the National Center for Childhood Diabetes at the Schneider Children's Medical Center of Israel (SCMCI) between December 2015 to December 2018. The inclusion criteria were a diagnosis of T1DM and using a CGM and an insulin pump simultaneously. Patients with insufficient data and pregnant or lactating women were excluded from the analysis. Table 1, below, depicts the clinical characteristics of the 141 patients included in the study. The average of CGM connection days was 64.4±46.6 per patient, summing up to a total of 9,083 CGM connection days and an overall of 1,592,506 glucose measurements that were included in the analysis.

The study protocol was approved by SCMCI institutional review board. Informed consent was waived by the institutional review board as all identifying details of the patients were removed prior to the computational analysis.

TABLE 1

| | |
|---|---|
| Number | 141 |
| Gender (F/M) | 66/75 |
| Weight (Kg) | 49.4 ± 14.6 |
| BMI (Kg/m²) | 20.5 ± 3.8 |
| Age (years) | 13.5 ± 5.2 |
| Duration of diabetes (years) | 5.2 ± 4.1 |
| Hemoglobin A1C (%) | 7.8 ± 1.0 |
| Celiac (Y/N) | 128/13 |
| Thyroid disorder (N/ | 126/12/4 |
| Hashimoto/Graves) | |
| Insulin Pump | Medtronic(74), Animas(19), Omnipod(48) |
| Continues glucose | Dexcom(48), Enlite(22), Guardian(24), |
| monitoring | Libre(43), Navigator(4) |

Data Preparation

As input for the computational models, 4 hours of historical CGM data and insulin dosage were used. Since some of the CGM devices report a glucose value every 15 minutes, linear interpolation was used to fill the missing BG values. The output metrics did not include interpolated data. Insulin dosage included both the bolus rate (which was considered as 0 if none) and the basal rate from the insulin pump records. The time-window of one-hour post insulin bolus administration was not predicted since a bolus is a very influential bidden feature that may affects the prediction accuracy. In addition, in order to model how much insulin is needed per meal, a future prediction of what is the expected BG level without such a bolus is required.

In Silico Data

In addition to the real-life data acquired, the neural network procedure of the present embodiments was also trained and tested on data generated by the distributed version of the UVA/Padova T1DM Simulator, which includes in silico data of 30 simulated T1DM patients (10 adults, 10 adolescents, and 10 children) (15). This simulator was accepted in 2008 by the US Food and Drug Administration (FDA) as a substitute for preclinical trials for insulin treatments, including closed-loop algorithms for AP systems. Each virtual subject in the simulator is represented with subject-specific model parameters. For each virtual subject 30 days of data were generated for training, 7 days were generated for validation, and 7 days were generated for testing. For children, 3 meals and 2 snacks were included for each day, while for adults, 3 meals and a single snack. Total carbohydrates consumption per day was calculated according to Dietary reference intake (DRI) recommendations (19). The input for the neural network procedure was 4 hours of historical CGM data and insulin dosing, comparable to the real-life data.

Machine Learning Models

It this Example, in addition to the gradually connected neural network procedure of the present embodiments, the following machine learning models were evaluated

Auto-Regressive model (AR) (20).

Tree ensemble using the Random Forest Regressor (RF) implementation of Scikit-Learn (21).

Gradient Boosting Decision Tree using LightGBM (22).

Fully connected neural networks using R(23).

For each model multiple hyperparameters were tested. All the models were tested using 10-fold cross validation.

Gradually Connected Network

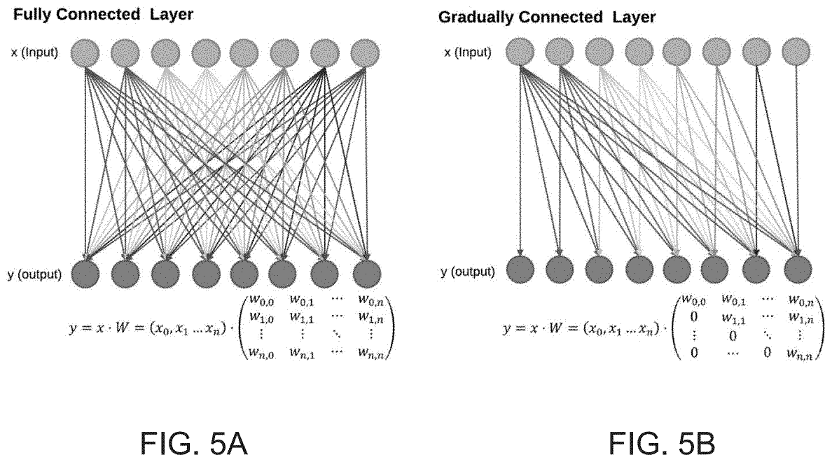

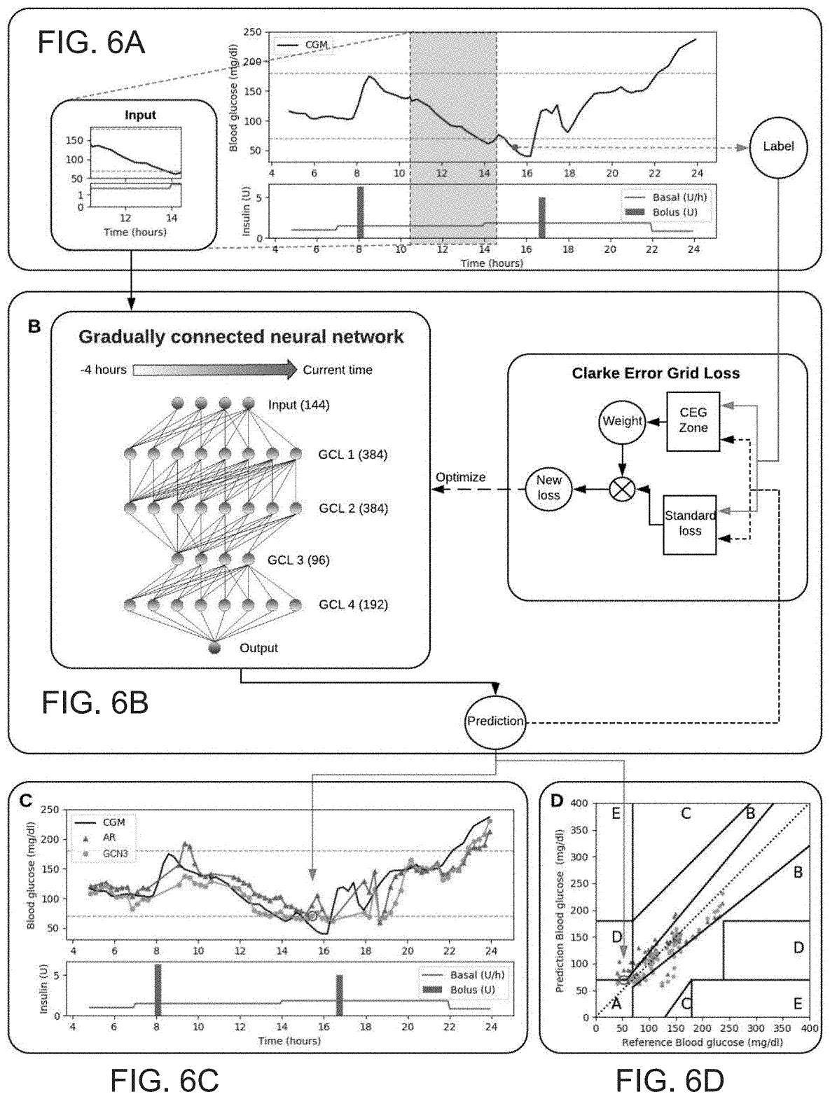

The Gradually connected network (GCN) was composed of Gradually connected layers (GCL) with fully connected layers on top (FIG. 5B, FIG. 6B). This is unlike traditional models which use only fully connected layers (FIG. 5A). GCL is similar to a fully connected layer, but with the number of connections to the output neurons gradually increasing with relation to the input order.

The GCL has 2 parameters “output_rows” and “step_size”, for input x∈M_{rows×columns} the output is y∈M_{rows×columns/step_size} where each cell in column i of the output is a linear combination of the first i·step_size columns of the input.

In a fully connected layer for input vector x=(x₀, x₁ . . . xₙ) and output vector y=(y₀, y₁ . . . yₘ) the weight matrix is W∈M_{n×m} where: y=x·W (FIG. 5A). In a GCL, W is an upper triangular block matrix (FIG. 5B).

In practice, the GCL was implemented by flattening the input, so the input is a vector of size (rows×columns), and then multiplying it with a weights matrix is a block upper triangular Matrix W∈M_{rows·columns×output_rows columns/step_size} where the height of the blocks is output_rows and the width of the blocks is (step_size·columns), so ∀c∈columns, W_{i<c·rows,j<output_rows c/step_size}=0.

GCN was found by the inventor very useful for sequential CGM data, since it gives more expressive power to more recent data (e.g., the last 30 minutes), which are more informative and more relevant for the prediction task. The GCN used in this Example contain 4 GCLs with 1-2 fully connected layers on top, summing up to a total of 4-5 hidden layers.

Models Optimization and Evaluation

The performance of the methods were optimized and evaluated using two performance measures: Numerical accuracy, measured using the root means square error (RMSE) and Clinical accuracy, measured by the percentage of prediction in zones C-E on the Clarke error grid (CEG) (18,24).

CEG is a method that quantifies clinical accuracy of predicted BG compared to reference BG, by classifying each pair of predicted and reference BG into five zones (FIG. 61)). Predictions that fall in the diagonal of the graph correspond to perfect agreement between the predicted BG and the reference BG, whereas points below and above the line indicate overestimation or underestimation of the actual BG values. While zones A and B indicate sufficiently accurate or acceptable errors in glucose value, zones C-E indicate unacceptable or potentially risky errors that may result in inappropriate treatment and undesired hypo- or hyperglycemia. Predictions in zones C-E, were therefore defined as “Clinically hazard zones” (CHZ). When training a neural network model, the use of different loss functions can affect the results of the model. The standard loss functions used in the present Example were the mean square error (MSE) and mean absolute percentage error (MAPE). In addition, a new loss functions was designed with the goal of minimizing the number of predictions in the CHZ of the CEG and optimizing the GCN to gain maximal clinical accuracy (defined as predictions in zones A-B). For each prediction, a standard loss (MSE or MAPE) was calculated and was multiplied by a weight according to the CEG zone it corresponded to before running the optimization algorithm. The weight for each zone was deterministically chosen and multiple options of different weights were examined.

To assess statistical significance, a t-test for comparisons between the models, and ANOVA between the performance of the same model on different subgroups were performed.

### Results

The GCN of the present embodiments, trained and tested on real-life data, achieved clinical accuracy of 99.3% and 95.8% in predicting glucose level 30 minutes and 60 minutes ahead, respectively, and reduced the percentage of glucose predictions in zones C, D and E of the CEG by 60.6% and 38.4% in these prediction horizons compared to a standard autoregressive model. The GCN of the present embodiments was superior to all other prediction models across all age groups and achieved higher clinical accuracy in subgroups of patients with high glucose variability and greater time spent in hypoglycemia. Compared to real-life data, when evaluated on in silico data, the GCN of the present embodiments had a higher clinical and numerical accuracy.

Comparing Different Computational Models

For each computational method, multiple models were trained using different hyperparameters for both the 30- and 60-minute prediction horizons (PH). The results were tested using 10-fold cross validation. The RMSE and percentage of time in the CHZ of the CEG was calculated for each method and every patient. Tables 2A and 2B, below, present the mean and standard deviation of each of these measurements using the best model of each method. The percentage of glucose predictions in CHZ using each of the models was compared to the baseline model. Table 2B is an extension of Table 2A, with the addition of the results of the mean absolute percentage error (MAPE) and the percentages of the predictions in each of the clarke error grid zones using the different computational methods.

In Tables 2A and 2B, Relative change was calculated by dividing the percentage of prediction in zones C-E on the CEG [C-E (%)] of the model, to the percentage of prediction in these zones when using a baseline AR model. AR—Autoregression model, CEG—Clarke Error Grid, FC—Fully connected neural network model, GCN—Gradually connected network, GCN1-3 are ensembles of several GCN models that were trained using different zone weights and standard loss function, LightGBM—LightGBM, Gradient Boosting Decision Tree, MAPE—mean absolute percentage error, PH—prediction horizon, RMSE—Root means square error, RF-Random Forest Regressor.

TABLE 2A
Performance of the computational models for
30 and 60 minutes glucose prediction horizons

| PH | | | | Relative |
|---|---|---|---|---|
| (minutes) | Model | RMSE | C-E (%) | change* |
| 30 | AR | 23.16 (3.80) | 1.87 (1.26) | 0.00% |
| | GCN3 | 24.27 (3.73) | 0.74 (0.56) | −60.64% |
| | GCN2 | 22.82 (3.81) | 1.20 (0.83) | −35.64% |
| | GCN1 | 22.06 (3.87) | 2.61 (1.75) | 39.29% |
| | FC | 22.85 (3.73) | 2.00 (1.32) | 6.72% |
| | LightGBM | 22.26 (3.60) | 2.97 (1.92) | 58.48% |
| | RF | 22.75 (3.72) | 2.95 (1.98) | 57.55% |
| 60 | AR | 39.94 (7.24) | 6.78 (5.14) | 0.00% |
| | GCN3 | 42.87 (8.12) | 4.18 (4.26) | −38.38% |
| | GCN2 | 39.69 (7.83) | 5.46 (4.58) | −19.51% |
| | GCN1 | 37.50 (6.94) | 7.28 (5.55) | 7.26% |
| | FC | 39.16 (6.91) | 6.66 (5.06) | −1.80% |
| | LightGBM | 37.34 (6.53) | 7.64 (5.81) | 12.68% |
| | RF | 38.97 (7.04) | 7.86 (5.82) | 15.83% |

TABLE 2B
Clinical and numerical accuracy of the computational model (10-fold cross validation)

| PH | | | | C-E | Relative | | | | | |
|---|---|---|---|---|---|---|---|---|---|---|
| (minutes) | Model | RMSE | MAPE | (%) | change* | A | B | C | D | E |
| 30 | AR | 23.16 ± 3.80 | 12.01 ± 3.20 | 1.87 ± 1.26 | 0.00% | 83.64% | 14.48% | 0.07% | 1.80% | 0.01% |
| | gcn3 | 24.27 ± 3.73 | 12.05 ± 3.73 | 0.74 ± 0.56 | −60.64% | 82.60% | 16.66% | 0.03% | 0.69% | 0.02% |
| | gcn2 | 22.82 ± 3.81 | 11.24 ± 3.81 | 1.20 ± 0.83 | 35.64% | 84.73% | 14.07% | 0.04% | 1.16% | 0.01% |
| | gcn1 | 22.06 ± 3.87 | 11.57 ± 3.87 | 2.61 ± 1.75 | 39.29% | 84.51% | 12.88% | 0.05% | 2.55% | 0.01% |
| | FC | 22.85 ± 3.73 | 11.93 ± 3.73 | 2.00 ± 1.32 | 6.72% | 83.72% | 14.29% | 0.05% | 1.94% | 0.01% |
| | LightGBM | 22.26 ± 3.60 | 11.84 ± 3.60 | 2.97 ± 1.92 | 58.48% | 84.07% | 12.97% | 0.03% | 2.93% | 0% |
| | RF | 22.75 ± 3.72 | 12.05 ± 3.72 | 2.95 ± 1.98 | 57.55% | 83.66% | 13.39% | 0.05% | 2.89% | 0% |
| 60 | AR | 39.94 ± 7.24 | 22.12 ± 7.24 | 6.78 ± 5.14 | 0.00% | 63.24% | 29.98% | 0.61% | 5.98% | 0.19% |
| | gcn3 | 42.87 ± 8.12 | 20.39 ± 8.12 | 4.18 ± 4.26 | 38.38% | 62.61% | 33.21% | 0.19% | 3.77% | 0.26% |
| | gcn2 | 39.69 ± 7.83 | 19.37 ± 7.83 | 5.46 ± 4.58 | −19.51% | 65.55% | 28.99% | 0.19% | 5.15% | 0.1% |
| | gcn1 | 37.50 ± 6.94 | 20.88 ± 6.94 | 7.28 ± 5.55 | 7.26% | 65.56% | 27.17% | 0.34% | 5.87% | 0.07% |
| | FC | 39.16 ± 5.91 | 21.78 ± 6.91 | 6.66 ± 5.06 | 1.80% | 63.48% | 29.85% | 0.45% | 5.05% | 0.16% |
| | LightGBM | 37.34 ± 6.53 | 21.26 ± 6.53 | 7.64 ± 5.81 | 12.68% | 64.81% | 27.54% | 0.32% | 7.26% | 0.06% |
| | RF | 38.97 ± 7.04 | 22.01 ± 7.04 | 7.86 ± 5.82 | 15.83% | 63.66% | 28.48% | 0.52% | 7.25% | 0.09% |

Tree-based methods, RF model and a Gradient Boosting Decision Tree model had better numerical accuracy than AR models. However, both models had lower clinical accuracy than AR.

The Fully connected neural network (FCN) model was next investigated with 2 hidden layers and width 50, which did not improve the numerical accuracy compared to RF and LightGBM but did improve clinical accuracy.

This Example uses ensembles of GCNs that were trained using different zone weights and standard loss functions. The first GCN ensemble (GCN1) is an ensemble of 2 GCNs, both using only MSE as the loss function (all zone weights are 1). This ensemble improved the RMSE result significantly compared to FC and achieved the lowest RMSE for 30 minutes PH and very similar RMSE result to the LightGBM for the 60 minutes PH. The second GCN (GCN2) ensembleoptimizes both numerical and clinical accuracy, and is an ensemble of 24 models with 6 unique zone weights, some of which focus on zones C-E of the CEG while others are more balanced and also give a higher weight to zones A-B. This ensemble resulted in an improvement of both clinical and numerical accuracy compared to AR. The third GCN ensemble (GCN3) optimized at least for decreasing the prediction in the CHZ of the CEG, and is an ensemble of 6 models which had weights for the different zones of the CEG, and uses both MSE and MAPE for the standard loss. This ensemble resulted in a relatively large improvement in clinical accuracy compared to AR, reducing the average percentage of predictions in zones C-E of the CEG relative to AR by 60% for 30 minutes PH and by 38% for 60 minutes PH. While GCN3 managed to significantly increase the clinical accuracy, it had the lowest numerical accuracy compared to all the other computational methods in both prediction horizons.

Comparison of GCN and the Autoregressive Model

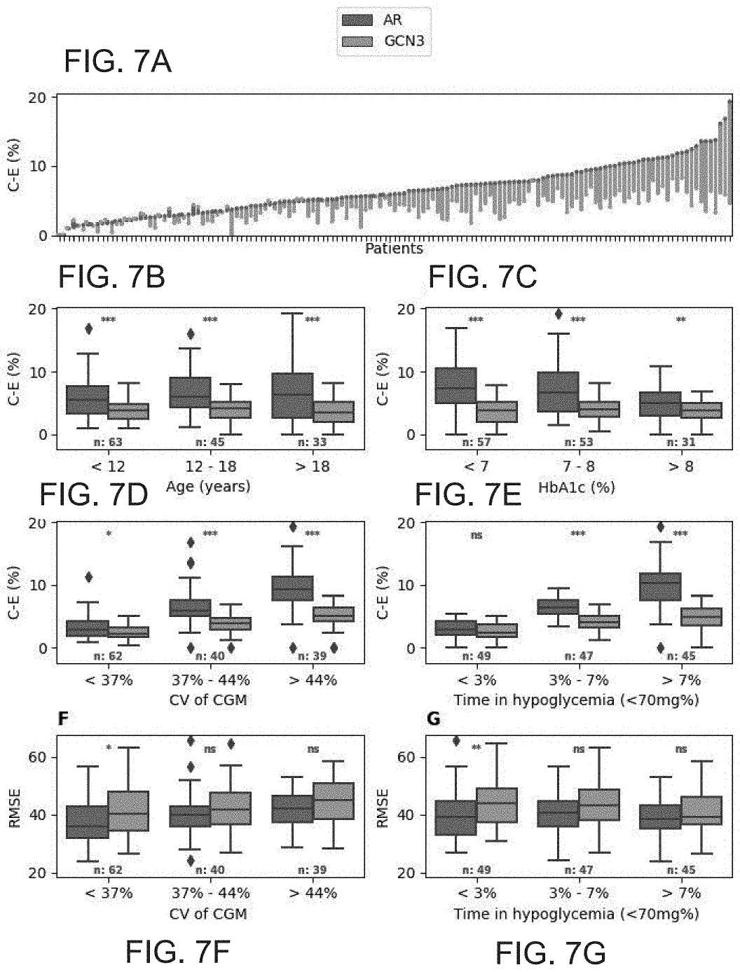

To further investigate the performance of the GCN of the present embodiments, several performance measures were analyzed for 60 minutes glucose PH on different subgroups of patients. These analyses are presented in FIGS. 7A-G and Tables 3 and 4, below. Tables 3 and 4 present the analysis of the performance of the models on different subgroups of patients, where Table 3 presents the mean value of each performance measure for each computational model, and Table 4 presents the comparison of mean values of each performance measure of the same computational model across different subgroups of patients. In Tables 3 and 4 hypoglycemia was defined as glucose level <70 mg/dl. In Table 3, significance between the performance of the two models on each subgroup was calculated using t-test, and in Table 4, significance was calculated using ANOVA. AR—Autoregression model, CV—Coefficient of variation, CGM—Continuous glucose monitoring, CEG—Clarke Error Grid, GCN3—Gradually connected network 3, PH—prediction horizon, RMSE—Root means square error, RF—Random Forest Regressor.

TABLE 3
Comparison between the computational models on subgroups of patients

| PH | | | | C-E (%) | | | RMSE | | |
|---|---|---|---|---|---|---|---|---|---|
| (minutes) | | Subgroups | | AR | GCN3 | Signficance | AR | GCN3 | Signficance |
| 30 | Age | <12 | y | 1.82 | 0.84 | <0.0001 | 24.35 | 25.53 | 0.0701 |
| | | 12-18 | y | 1.95 | 0.71 | <0.0001 | 22.37 | 23.53 | 0.0936 |
| | | >18 | y | 1.86 | 0.66 | <0.0001 | 22.96 | 24.05 | 0.2944 |
| | HbA1c | <7% | | 2.34 | 0.8 | <0.0001 | 23.4 | 24.48 | 0.4032 |
| | | 7%-8% | | 1.91 | 0.75 | <0.0001 | 22.94 | 24.07 | 0.0352 |
| | | >8% | | 1.59 | 0.69 | <0.0001 | 23.21 | 24.42 | 0.1115 |
| | CV of CGM | <37% | | 0.96 | 0.46 | 0.0005 | 22.18 | 23.76 | 0.0581 |
| | | 37%-45% | | 1.97 | 0.78 | <0.0001 | 23.43 | 24.23 | 0.2552 |
| | | >45% | | 2.69 | 0.96 | <0.0001 | 23.67 | 24.94 | 0.1283 |
| | Percentage of | <3% | | 0.77 | 0.42 | <0.0001 | 23.58 | 24.95 | 0.1066 |
| | time spent in | 3%-7% | | 2 | 0.83 | <0.0001 | 23.25 | 24.3 | 0.137 |
| | hypoglycemia | >7% | | 2.97 | 1 | <0.0001 | 22.55 | 23.56 | 0.1958 |
| 60 | Age | <12 | y | 5.99 | 3.89 | 0.0002 | 40.38 | 43.09 | 0.0375 |
| | | 12-18 | y | 6.74 | 4.01 | <0.0001 | 40.6 | 43.72 | 0.0459 |
| | | >18 | y | 6.63 | 3.52 | 0.0005 | 37.8 | 40.84 | 0.1032 |
| | HbA1c | <7% | | 7.56 | 3.66 | <0.0001 | 36.85 | 37.94 | 0.6186 |
| | | 7%-8% | | 7.11 | 3.99 | <0.0001 | 39.03 | 41.92 | 0.0194 |
| | | >8% | | 5.17 | 3.82 | 0.0012 | 42.55 | 46.71 | 0.0029 |
| | CV of CGM | <37% | | 3.32 | 2.55 | 0.0399 | 37.34 | 41.96 | 0.0211 |
| | | 37%-45% | | 6.62 | 3.91 | <0.0001 | 40.11 | 42.43 | 0.0788 |
| | | >45% | | 9.49 | 5.11 | <0.0001 | 42.09 | 44.41 | 0.1349 |
| | Percentage of | <3% | | 3.06 | 2.63 | 0.1134 | 39.91 | 44.47 | 0.0099 |
| | time spent in | 3%-7% | | 6.59 | 4.1 | <0.0001 | 40.5 | 43.36 | 0.0501 |
| | hypoglycemia | >7% | | 10.08 | 4.93 | <0.0001 | 39.18 | 40.54 | 0.3745 |

TABLE 4
Comparison of the performance of the same computational
model across different subgroups of patients

| | | | C-E (%) | | RMSE | |
|---|---|---|---|---|---|---|
| PH | | | Signficance | | Signficance | |
| (minutes] | | Subgroups | AR | GCN3 | AR | GCN3 |
| 30 | Age | <12 y,12-18 y,>18 y | 0.5464 | 0.4463 | 0.1672 | 0.2525 |
| | HbA1c | <7%, 7 %-8%, >8% | 0.0028 | 0.7106 | 0.0009 | <0.0001 |
| | CV of CGM | <37%, 37%-45%, >45% | <0.0001 | <0.0001 | 0.0121 | 0.3569 |
| | Percentage of time spent | <3%, 3%-7%, >7% | 4.0001 | <0.0001 | 0.6834 | 0.0557 |
| | in hypoglycemia | | | | | |
| | Age | <12 y, 12-18 y, >18 y | 0.8588 | 0.345 | 0.0262 | 0.0204 |
| | HbA1c | <7%, 7%-8%, >8% | 0.0285 | 0.6743 | 0.8491 | 0.8479 |
| | CV of CGM | <37%, 37%-45%, >45% | 0.0001 | 0.0001 | 0.1624 | 0.3717 |
| | Percentage of fee spent | <3%, 3%-7%, >7% | <0.0001 | <0.0001 | 0.4178 | 0.1962 |
| | in hypoglycemia | | | | | |

An overall analysis showed that the GCN of the present embodiments decreased the percentage of predictions in CHZ of the CEG significantly (p<10′). Examining the individual level, the decrease was apparent in the majority of the cohort (123 of 141, 87%; (FIG. 7A). The percentage of predictions in these zones was not significantly different between different age groups (p=0.45 in GCN3 and p=0.58 for AR model) (FIG. 7B).

To examine if our The GCN of the present embodiments performs differently on patients with a different degree of glycemic control, data on HbA1c was used and 3 subgroups were created: patients with good glycemic control (HbA1C<7%), moderate glycemic control (7%<HbA1C<8%), and poor glycemic control (HbA1C>8%) (FIG. 7C). The clinical accuracy of the GCN3 was not affected by HbA1C level (p=0.69), while the clinical accuracy of the AR model significantly decreased for patients with lower HbA1C level, reflected by an increase percentage of predictions in the CHZ zones of the CEG (p=0.006).

Subgroups of patients with different percentage of time spent in hypoglycemia, defined as a glucose level less than 70 mg/dl were also investigated. The cohort was divided into 3 groups, in which percentage of time spent in hypoglycemia is less than 3%, between 3%-7% and above 7%. In both models there was a higher average percentage of predictions in the CHZ of the CEG when the percentage of time spent in hypoglycemia increased. In the high hypoglycemic risk group of patients, the GCN decreased the average percentage of predictions in the CHZ of the CEG compared to the AR mode by 66.4% for 30 minutes PH (2.91% using AR vs. 0.98% using GCN3) and by 46% for 60 minutes PH (10.95% using AR vs. 5.91% using GCN3 (p<10⁻¹² for both), demonstrating higher clinical accuracy for the GCN of the present embodiments in this high risk population. Both models had a small decrease (0.7) in the average RMSE in patients that spend a large percentage of time in hypoglycemia (above 7%). (FIGS. 7E and 7G, and Table 3).

To study the effect of glucose variability on the performance of the GCN of the present embodiments, the Coefficient of variation (CV) (26,27) was calculated from the CGM measurements of each patient. This value measures blood glucose variability corrected for the mean blood glucose per patient. The cohort was divided into 3 subgroups according to their CV: below 37%, between 37% and 45%, and above 45%. The average percentage of predictions in the CHZ of the CEG and average RMSE monotonically increased with an increase in the CV using both models, reflecting the challenge of BG prediction in patients with high BG variability. Even in the group of patients with the highest CV (larger than 45%), the GCN of the present embodiments decreased the average percentage of predictions in the CHZ of the CEG by 66.6% for 30 minutes PH (2.82% using AR vs. 0.94% using GCN3) and by 40.9% for 60 minutes PH (11.63% using AR vs. 6.87% using GCN3) (p<10⁻⁸ for both), reinforcing the significantly better clinical accuracy of the GCN of the present embodiments in patients with high blood glucose variability (FIGS. 7D and 7F).

Analysis Using the Type 1 Diabetes Simulator

Similar to the analysis done on real-life data, the GCN and other models were trained for two glucose PH, 30 and 60 minutes, using different hyperparameters, on data generated by the UVA/Padova Type 1 Diabetes Simulator. The results were tested on 7 generated days for each patient and calculated the RMSE and percentage of time in the CHZ of the CEG for each method per patient. In order to compare the results on simulated data versus real-life data, the GCN and other models were trained on the real-life dataset of 141 T1DM patients, reserving the last 4 days of each patient for testing.

Table 5 presents the results on a models trained using all the real-life data of T1DM patients and tested on the last 4 days of each patient (that were not included in the training) Relative change was calculated by dividing the percentage of prediction in zones C-E on the CEG [C-E (%)] of the model, to the percentage of prediction in these zones when using a baseline AR model. AR—Autoregression model, CEG—Clarke Error Grid, FC—Fully connected neural network model, GCN—Gradually connected network, GCN1-3 ensembles of several GCN models that were trained using different zone weights and standard loss function, LightGBM—LightGBM, Gradient Boosting Decision Tree, MAPE—mean absolute percentage error, PH—prediction horizon, RMSE—Root means square error, RF—Random Forest Regressor.

TABLE 5
Clinical and numerical accuracy of the computational models (tested on the last 4 days of each patient)

| PH | | | | CE | Relative | | | | | |
|---|---|---|---|---|---|---|---|---|---|---|
| (minutes) | Model | RMSE | MAPE | (%) | change* | A | B | C | D | E |
| 30 | AR | 22.72 ± 6.31 | 11.83 ± 6.31 | 1.97 ± 2.06 | 0.00% | 83.86% | 14.18% | 0.10% | 1.85% | 0.01% |
| | gcn3 | 23.69 ± 7.23 | 11.77 ± 7.23 | 0.77 ± 1.09 | −61.00% | 84.06% | 15.17% | 0.05% | 0.70% | 0.02% |
| | gcn2 | 22.29 ± 6.82 | 11.06 ± 6.82 | 1.27 ± 1.58 | −35.60% | 85.38% | 13.35% | 0.04% | 1.20% | 0.02% |
| | gcn1 | 21.49 ± 6.44 | 11.17 ± 6.44 | 2.30 ± 2.63 | 17.26% | 85.23% | 12.47% | 0.05% | 2.24% | 0.01% |
| | FC | 22.68 ± 6.32 | 11.50 ± 6.32 | 1.75 ± 1.86 | −11.12% | 84.72% | 13.54% | 0.04% | 1.69% | 0.02% |
| | LightGBM | 22.09 ± 5.85 | 11.75 ± 5.85 | 3.05 ± 3.00 | 55.26% | 83.77% | 13.18% | 0.04% | 3.00% | 0% |
| | RF | 22.20 ± 5.98 | 11.77 ± 5.98 | 2.93 ± 3.02 | 49.32% | 83.77% | 13.30% | 0.07% | 2.86% | 0% |
| 60 | AR | 39.13 ± 10.43 | 22.08 ± 10.43 | 7.21 ± 9.65 | 0.00% | 62.80% | 29.99% | 0.68% | 6.37% | 0.16% |
| | gcn3 | 41.31 ± 13.14 | 19.72 ± 13.14 | 4.06 ± 4.00 | −43.65% | 64.95% | 30.99% | 0.23% | 3.61% | 0.23% |
| | gcn2 | 38.17 ± 11.89 | 18.97 ± 11.89 | 5.71 ± 9.18 | −20.77% | 66.39% | 27.90% | 0.22% | 5.39% | 0.10% |
| | gcn1 | 36.23 ± 10.82 | 20.11 ± 10.82 | 7.48 ± 10.08 | 3.78% | 66.86% | 25.66% | 0.35% | 7.05% | 0.09% |
| | FC | 38.60 ± 10.71 | 21.03 ± 10.71 | 6.68 ± 9.37 | −7.41% | 64.41% | 28.91% | 0.40% | 6.10% | 0.17% |
| | LightGBM | 37.03 ± 9.64 | 21.47 ± 9.64 | 8.16 ± 10.31 | 13.19% | 63.99% | 27.85% | 0.38% | 7.71% | 0.07% |
| | RF | 37.95 ± 9.92 | 21.86 ± 9.92 | 8.24 ± 10.29 | 14.32% | 63.31% | 28.45% | 0.57% | 7.62% | 0.05% |

Table 6 presents the results of the computational models on In-silico data generated by the data generated by the distributed version of the UVA/Padova Type 1 Diabetes Simulator, For each virtual subject we generated 30 days of data for training, 7 days for validation and 7 days for testing. When compared to the results obtained using real-life data (Table 5), these results are better, especially on 60 min PH, both in percentage of glucose prediction in CHZ and in percentage of glucose prediction in zone A of the CEG. Relative change was calculated by dividing the percentage of prediction in zones C-E on the CEG [C-E (%)] of the model, to the percentage of prediction in these zones when using a baseline AR model. AR—Autoregression model, CEG—Clarke Error Grid, FC—Fully connected neural network model, GCN—Single gradually connected network, optimized using MSE loss, LightGBM—LightGBM, Gradient Boosting Decision Tree, MAPE—mean absolute percentage error, PH—prediction horizon, RMSE—Root means square error, RF—Random Forest Regressor.

TABLE 6
Table S3
Clinical and numerical accuracy of the computational models on In silico data

| PH | | | | C-E | Relative | | | | | |
|---|---|---|---|---|---|---|---|---|---|---|
| (minutes) | Model | RMSE | MAPE | (%) | change* | A | B | C | D | E |
| 30 | AR | 21.42 | 11.83 | 1.28 | 0.00 | 85.46% | 13.26% | 0.02% | 1.26% | 0.01% |
| | FC | 15.68 | 8.51 | 0.94 | −26.56 | 93.45% | 5.61% | 0% | 0.93% | 0% |
| | GCN | 15.85 | 8.28 | 0.74 | 42.19 | 93.79% | 5.47% | 0.01% | 0.73% | 0% |
| | LightGBM | 15.53 | 7.94 | 1.02 | −20.31 | 94.02% | 4.96% | 0.01% | 1.01% | 0% |
| | RF | 17.64 | 9.45 | 1.38 | 7.81 | 91.60% | 7.01% | 0.01% | 1.37% | 0% |
| 60 | AR | 37.02 | 16.59 | 3.35 | 0.00 | 73.55% | 23.10% | 0.04% | 3.26% | 0.06% |
| | FC | 30.09 | 14.21 | 2.88 | −14.03 | 79.77% | 17.35% | 0.06% | 2.79% | 0.03% |
| | GCN | 30.46 | 13.21 | 2.44 | −27.16 | 82.31% | 15.25% | 0.12% | 2.26% | 0.06% |
| | LightGBM | 30.39 | 14.07 | 3.33 | −0.60 | 80.03% | 16.63% | 0.06% | 3.23% | 0.04% |
| | RF | 32.64 | 15.6 | 3.8 | 13.43 | 76.39% | 19.80% | 0.04% | 3.73% | 0.04% |

For both PH, all of the models had a better performance of clinical and numerical accuracy, as reflected by both a lower RMSE and percentage of time in CHZ of the CEG when trained and tested on the simulated data. For example, when trained and tested on real-life data, the highest percentage of prediction in zone A of the CEG for PH 60 was 66.8% on real-life data, and 82.3%, on the simulated data.

### DISCUSSION

This Example demonstrated a neural network procedure which was trained using BG measurements and insulin dosage, and which significantly improves blood glucose predictions at 30 and 60 minutes ahead compared to the commonly used AR model. The GCN of the present embodiments, optimized for clinical accuracy, reduces prediction errors that are considered hazardous and may lead to inappropriate clinical decisions.

In This Example, three GCNs were created, with different optimizations for clinical and numerical accuracies. The GCN3 model, optimized for maximal clinical accuracy, using the zones of the CEG in the loss function, achieved clinical accuracy of 99.3% and 95.8% in predicting glucose level 30 minutes and 60 minutes ahead respectively, and reduced the percentage of glucose predictions in the CHZ zones of the CEG by 60.6% and 38.4% in these prediction horizons compared to the commonly used AR model. The clinical accuracy of the GCN of the present embodiments was pronounced in specific clinical settings, in which the current models perform poorly, such as patients at high risk for hypoglycemia or those with increased glucose variability.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### REFERENCES

1. Patterson C C, Dahlquist G G, Gyürüs E, Green A, Soltész G, EURODIAB Study Group. Incidence trends for childhood type 1 diabetes in Europe during 1989-2003 and predicted new cases 2005-20: a multicentre prospective registration study. Lancet. 2009 Jun. 13; 373(9680):2027-33.
2. Quinn M, Fleischman A, Rosner B, Nigrin D J, Wolfsdorf J I. Characteristics at diagnosis of type 1 diabetes in children younger than 6 years. J Pediatr. 2006 March;148(3):366-71.
3. Diabetes Control and Complications Trial Research Group, Nathan D M, Genuth S, Lachin J, Cleary P, Crofford O, et al. The effect of intensive treatment of diabetes on the development and progression of long-term complications in insulin-dependent diabetes mellitus. N Engl J Med. 1993 Sep. 30; 329(14):977-86.
4. Hypoglycemia in the diabetes control and complications trial. the diabetes control and complications trial research group. Diabetes. 1997 February; 46(2):271-86.
5. Facchinetti A. Continuous glucose monitoring sensors: past, present and future algorithmic challenges. Sensors Basel Sensors. 2016 Dec. 9; 16(12).
6. Wood J R, Miller K M, Maahs D M, Beck R W, DiMeglio L A, Libman I M, et al. Most youth with type 1 diabetes in the T1D Exchange Clinic Registry do not meet American Diabetes Association or International Society for Pediatric and Adolescent Diabetes clinical guidelines. Diabetes Care. 2013 July; 36(7):2035-7.
7. Petitti D B, Klingensmith G J, Bell R A, Andrews J S, Dabelea D, Imperatore G, et al. Glycemic control in youth with diabetes: the SEARCH for diabetes in Youth Study. J Pediatr. 2009 November; 155(5):668-72.
8. Miller K M, Foster N C, Beck R W, Bergenstal R M, DuBose S N, DiMeglio L A, et al. Current state of type 1 diabetes treatment in the U.S.: updated data from the T1D Exchange clinic registry. Diabetes Care. 2015 June; 38(6):971-8.
9. Hovorka R. Closed-loop insulin delivery: from bench to clinical practice. Nat Rev Endocrinol. 2011 Feb. 22; 7(7):385-95.
10. Cobelli C, Renard E, Kovatchev B. Artificial pancreas: past, present, future. Diabetes. 2011 November; 60(11):2672-82.
11. Magni L, Raimondo D M, Bossi L, Man C D, De Nicolao G, Kovatchev B, et al. Model predictive control of type 1 diabetes: an in silico trial. J Diabetes Sci Technol. 2007
12. Doyle F J, Huyett L M, Lee J B, Zisser H C, Dassau E. Closed-loop artificial pancreas systems: engineering the algorithms. Diabetes Care. 2014; 37(5):1191-7.
13. Atlas E, Nimri R, Miller S, Grunberg E A, Phillip M. MD-logic artificial pancreas system: a pilot study in adults with type 1 diabetes. Diabetes Care. 2010 May; 33(5):1072-6.
14. Man C D, Micheletto F, Lv D, Breton M, Kovatchev B, Cobelli C. The UVA/PADOVA type 1 diabetes simulator: new features. J Diabetes Sci Technol. 2014 Jan. 1; 8(1):26-34.
15. Kovatchev B P, Breton M, Man C D, Cobelli C. In silico preclinical trials: a proof of concept in closed-loop control of type 1 diabetes. J Diabetes Sci Technol. 2009 January; 3(1):44-55.
16. Krizhevsky A, Sutskever I, Hinton G E. ImageNet classification with deep convolutional neural networks. Commun ACM. 2012 May 24; 60(6):84-90.
17. Hinton G, Deng L, Yu D, Dahl G, Mohamed A, Jaitly N, et al. Deep Neural Networks for Acoustic Modeling in Speech Recognition. IEEE Signal Processing Magazine. 2012 Nov. 1
18. Clarke W L, Cox D, Gonder-Frederick L A, Carter W, Pohl S L. Evaluating clinical accuracy of systems for self-monitoring of blood glucose. Diabetes Care. 1987 October; 10(5):622-8.
19. Nutrient Recommendations: Dietary Reference Intakes (DRI) [Internet]. [cited 2019 Mar. 14]. Available from: www(dot)ods(dot)od (dot)nih (dot)gov/Health_Information/Dietary_Reference_Intakes.aspx
20. Leal Y, Garcia-Gabin W, Bondia J, Esteve E, Ricart W, Fernandez-Real J-M, et al. Real-time glucose estimation algorithm for continuous glucose monitoring using autoregressive models. J Diabetes Sci Technol. 2010 Mar. 1; 4(2):391-403.
21. Pedregosa F, Varoquaux G, Gramfort A, Michel V, Thirion B, Grisel O, et al. Scikit-learn: Machine Learning in Python. Journal of Machine Learning Research. 2011;
22. Ke G, Meng Q, Finley T, Wang T, Chen W, Ma W, et al. LightGBM: A Highly Efficient Gradient Boosting Decision Tree. 2017;
23. Chollet F. Keras., GitHub. www(dot)github(dot)com/fchollet/keras. 2015;
24. Kovatchev B, Anderson S, Heinemann L, Clarke W. Comparison of the numerical and clinical accuracy of four continuous glucose monitors. Diabetes Care. 2008 June; 31(6):1160 4.
25. American Diabetes Association. 6. Glycemic Targets: Standards of Medical Care in Diabetes-2018. Diabetes Care. 2018; 41(Suppl 1):555-64.
26. Suh S, Kim J H. Glycemic variability: how do we measure it and why is it important? Diabetes Metab J. 2015 August; 39(4):273-82.
27. DeVries J H. Glucose variability: where it is important and how to measure it. Diabetes. 2013 May; 62(5):1405-8.
28. Zecchin C, Facchinetti A, Sparacino G, Cobelli C. Jump neural network for online short-time prediction of blood glucose from continuous monitoring sensors and meal information. Comput Methods Programs Biomed. 2014; 113(1):144-52.
29. Pérez-Gandia C, Facchinetti A, Sparacino G, Cobelli C, Gomez E J, Rigla M, et al. Artificial neural network algorithm for online glucose prediction from continuous glucose monitoring. Diabetes Technol Ther. 2010 January; 12(1):81-8.
30. Pappada S M, Cameron B D, Rosman P M. Development of a neural network for prediction of glucose concentration in type 1 diabetes patients. J Diabetes Sci Technol. 2008 September; 2(5):792-801.
31. Pappada S M, Cameron B D, Rosman P M, Bourey R E, Papadimos T J, Olorunto W, et al. Neural network-based real-time prediction of glucose in patients with insulin-dependent diabetes. Diabetes Technol Ther. 2011 February; 13(2):135-41.
32. Mougiakakou S G, Nikita K S. A neural network approach for insulin regime and dose adjustment in type 1 diabetes. Diabetes Technol Ther. 2000; 2(3):381-9.
33. Andelin M, Kropff J, Matuleviciene V, Joseph J I, Attvall S, Theodorsson E, et al. Assessing the accuracy of continuous glucose monitoring (CGM) calibrated with capillary values using capillary or venous glucose levels as a reference. J Diabetes Sci Technol. 2016 July; 10(4): 876-84.
34. Luijf Y M, Mader J K, Doll W, Pieber T, Farret A, Place J, et al. Accuracy and reliability of continuous glucose monitoring systems: a head-to-head comparison. Diabetes Technol Ther. 2013 August; 15(8):722-7.

1. A method of predicting an analyte level in a biological liquid, the method comprising:
receiving a time-ordered series of levels of the analyte, monitored over a time-period;
feeding a trained neural network procedure with said monitored levels; and
displaying, based on an output received from said procedure, a predicted level of the analyte in a future time;
wherein said procedure comprises a plurality of layers, and wherein for at least one pair of layers, a number of inter-layer connections within said pair is higher for later monitored levels than for earlier monitored levels.

2. The method according to claim 1, wherein the analyte comprises glucose.

3. The method of claim 1, comprising receiving time-ordered series of dose levels of a drug administered to the biological liquid during said time-period, and feeding said dose levels to said procedure.

4. The method of claim 1, wherein said time-period is selected such that said dose levels include basal dose levels but not bolus dose levels.

5. The method according to claim 3, wherein said future time is before administration of a bolus dose level of said drug.

6. The method according to claim 3, wherein the analyte comprises glucose and the drug comprises insulin.

7. (canceled)

8. The method according to claim 1, wherein said time-ordered series is characterized by a frequency of at least 6 analyte levels per hour.

9. (canceled)

10. The method according to claim 1, wherein said time-ordered series is characterized by a frequency of less than four analyte levels per hour, and the method comprises interpolating said time-ordered series to provide a plurality of interpolated analyte levels, and updating said time-ordered series using said interpolated analyte levels.

11. (canceled)

12. A computer software product, comprising a computer-readable medium in which program instructions are stored, which instructions, when read by a data processor, cause the data processor to receive a time-ordered monitored levels of the analyte over a time-period and to execute the method according to claim 1.

13. (canceled)

14. A system for predicting an analyte level in a biological liquid, the system comprising a monitoring device configured for monitoring levels of the analyte in the biological liquid over a time-period, a data processor, and a communication device configured for transmitting said monitored levels to said data processor in a time-ordered manner;
said data processor being configured for receiving said monitored levels, for accessing computer-readable medium storing a trained neural network procedure, for feeding said procedure with said monitored levels, and for displaying, based on output received from said procedure, a predicted level of the analyte in a future time;
wherein said procedure comprises a plurality of layers, and wherein for at least one pair of layers a number of inter-layer connections within said pair is higher for later monitored levels than for earlier monitored levels.

15. (canceled)

16. The system of claim 14, comprising an automatic drug administering device configured for administered a drug to the biological liquid during said time-period, and for transmitting to said data processor data pertaining to dosage of said administered drug as a time-ordered series of dose levels, wherein said data processor is configured for feeding said dose levels to said procedure.

17-22. (canceled)

23. The system according to claim 14, wherein said time-ordered series is characterized by a frequency of less than four analyte levels per hour, and the data processor is configured for interpolating said time-ordered series to provide a plurality of interpolated analyte levels, and updating said time-ordered series using said interpolated analyte levels.

24. (canceled)

25. The system according to claim 14, wherein said communication device communicates wirelessly with said data processor.

26. (canceled)

27. The system according to claim 25, wherein said data processor is a component of a server computer, and is configured to transmit said predicted level of the analyte to a mobile device having a display for displaying said predicted level of the analyte on said display.

28. The system according to claim 14, wherein said data processor is a component of a mobile device having a display, and wherein said data processor is configured to display said predicted level of the analyte on said display.

29. (canceled)

30. The method according to claim 1, wherein said time-period is from about 1 hour to about 6 hours.

31. (canceled)

32. The method according to claim 1, wherein said future time is at least 10 minutes after an end of said time-period.

33. (canceled)

34. The method according to claim 1, wherein said inter-layer connections are defined over a triangular weight matrix.

35. (canceled)

36. The method according to claim 1, wherein at least one layer of said procedure, other than said at least one pair of layers, is a fully connected layer.

37. (canceled)

38. The method according to claim 1, wherein at least one layer of said pair is a hidden layer.

39. (canceled)
